(19) United States
(12) Patent Application Publication
(10) Pub. No.: US 2011/0104127 A1
(43) Pub. Date: May 5, 2011

(54) mRNA-TRANSFECTION OF ADULT PROGENITOR CELLS FOR SPECIFIC TISSUE REGENERATION

(75) Inventors: Jan Torzewski, Immenstadt (DE); Vinzenz Hombach, Ulm (DE); Juliane Ingeborg Marie Wiehe, Blaubeuren (DE); Jochen Greiner, Sonnenbuhl (DE); Michael Schmitt, Breitingen (DE); Markus Wiesneth, Ulm (DE); Oliver Zimmermann, Westerstetten (DE); Hubert Schrezenmeier, Ulm (DE)

(73) Assignee: UNIVERSITAT ULM, Ulm (DE)

(21) Appl. No.: 12/278,952

(22) PCT Filed: Feb. 8, 2007
(86) PCT No.: PCT/EP07/01085

§ 371 Date: Dec. 21, 2010

(30) Foreign Application Priority Data
Feb. 8, 2006 (DE) 10 2006 005 827.5

(51) Int. Cl. A61K 35/12; C12N 5/10; A61P 9/00; A61P 17/00; A61P 1/00; A61P 13/00; A61P 19/00

(52) U.S. Cl. 424/93.21; 435/325; 435/366

(57) ABSTRACT

The present invention relates to progenitor cells, medicaments containing progenitor cells and also uses thereof for specific tissue regeneration. Such processes are required in all medical sectors, in particular in the treatment of cardiovascular, haematological, nephrological, neurological, dermatological, gastroenterological or orthopaedic disorders. The progenitor cells are characterized in that they are transfected with mRNA which codes for a protein which promotes colonization of the progenitor cells in a target tissue and/or the differentiation of the progenitor cells in target cells or target tissue cells.

The present invention relates to progenitor cells, pharmaceutical products containing progenitor cells and their use for specific tissue regeneration. Methods of this type are needed in all areas of medicine, in particular in the treatment of cardiovascular, hematological, nephrological, neurological, dermatological, gastrointestinal or orthopedic disorders.

It is known from the prior art that cells can be transfected by means of mRNA. To date, such transfected cells have been used in tumor therapy to introduce suitable transfected cells into the tumor tissue. This makes it possible to mark certain tissue types and thus to make them accessible to targeted tumor therapy.

mRNA transfection techniques suitable for this purpose have been described, for example, by Smits et al., Leukemia 2004, pages 1-5, “RNA-based gene transfer for adult stem cells and T cells.”

The present invention uses these known transfection methods as a starting point and intends to make available methods and substances as well as pharmaceutical products and methods to produce such pharmaceutical products, by means of which it is possible to regenerate tissue.

This objective is reached with the progenitor cells as in Claim 1, with the pharmaceutical product as in Claim 2, with the use of the progenitor cells as in Claims 3 and 4, and with the therapeutic and nontherapeutic methods and production methods following the claims. Useful improvements follow from the dependent claims.

The present invention takes advantage of the fact that mRNA transfection methods are already known from the prior art and have been successfully used in tumor therapy. Using this prior art as a starting point, the present invention builds on the idea underlying the invention and on the knowledge that progenitor cells can be transfected with mRNAs that code for a protein which promotes homing of the transfected progenitor cells to a specific target tissue and/or the differentiation of the transfected progenitor cells in cells of a specific type of target tissue. Thus, it is now possible for the first time to introduce the progenitor cells into a specific target tissue and promote homing and/or to specifically produce target cells which can subsequently be used for different purposes.

In contrast to other conventional gene-technological methods, mRNA transfection is not subject to the strict rules of law that apply to genetically altered cells. The reason is that the transfected cell is not genetically altered by mRNA transfection but that instead it merely produces the protein that was coded by the mRNA. Within the transfected cell, the mRNA is rapidly degraded so that after a short time, the cell returns to its original state.

The present invention takes advantage of this mechanism in that the progenitor cells are appropriately transfected so that they are enabled for a short period of time to differentiate into specific target cells or to home in on a specific target tissue. After degradation of the introduced mRNA and of the protein which was coded by this mRNA, the resultant cell is unaltered and in autologous progenitor cells does not differ from the cells of the target tissue.

In this context, progenitor cells include all cells not yet terminally differentiated, in particular hematopoietic progenitor cells, neuronal progenitor cells, progenitor cells of the liver, of the skeletal muscle and of the skin as well as progenitor cells from the blood of the umbilical cord. The progenitor cells especially preferably used are stem cells, in particular stem cells of nonembryonal origin, i.e., adult stem cells, tissue-specific adult stem cells and other not yet fully differentiated cells.

Blau et al., in Cell, volume 5, pp. 829-841 (2001), “The evolving concept of a stem cell: Entity or function,” offer a conspectus of the progenitor cells that can be used in the present invention.

Because of the possibility of tissue regeneration and the production of differentiated target cells of a specific target tissue, the method according to the present invention is highly suitable for use in the treatment of cardiovascular, hematological, nephrological, neurological disorders, skin disorders, gastrointestinal disorders and/or orthopedic disorders in which tissue is to be regenerated. The method according to the present invention and the cells or pharmaceutical products according to the present invention can also be used to treat other clinical syndromes.

A list which is not conclusive but offers only examples of disorders to be treated follows; this list also includes the treatment mechanism and the transfecting mRNA to be used:

### 1. Cardiovascular Disorders

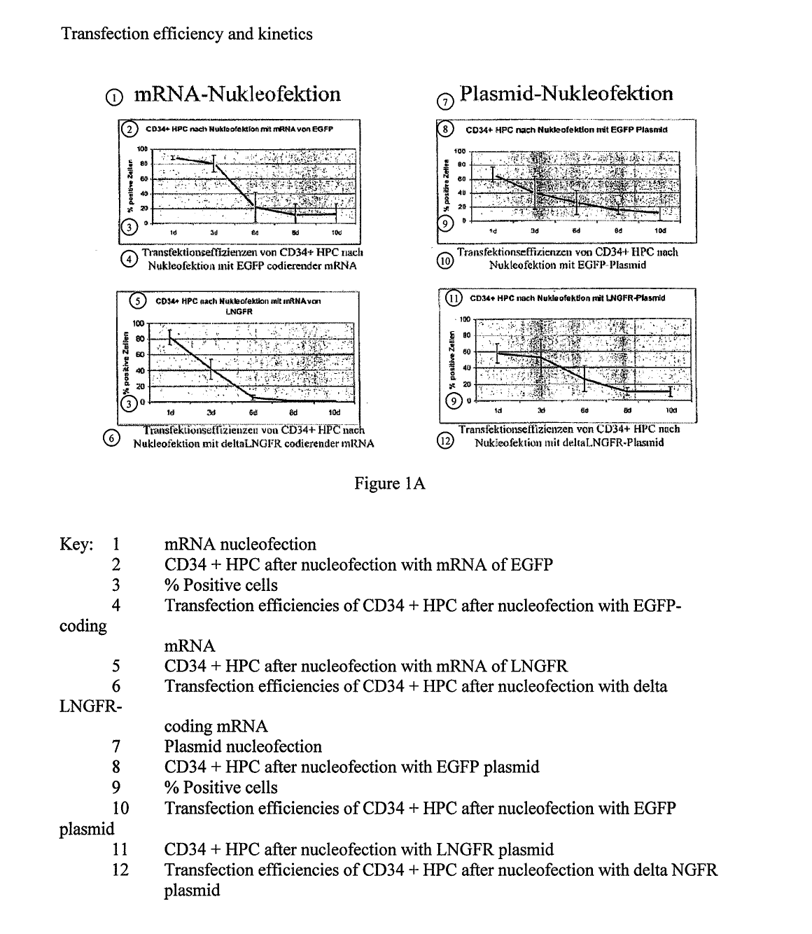

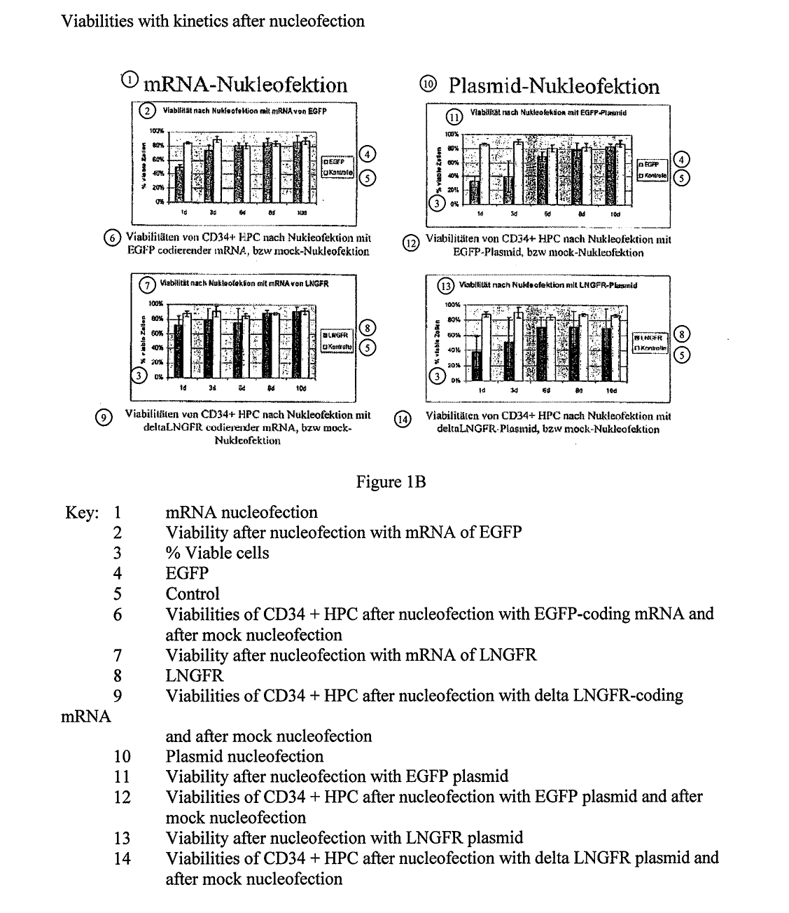

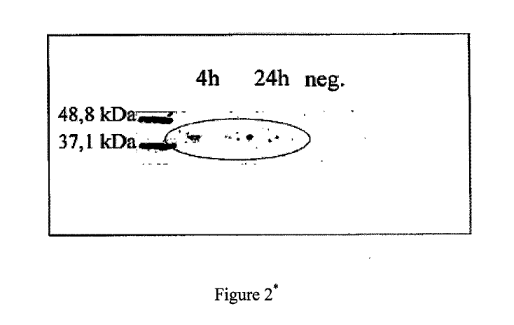

e.g., myocardial infarction: intravasal or intramyocardial administration of mRNA-transfected progenitor cells, such as CD34-positive progenitor cells or mesenchymal stem cells, see FIGS. 1 and 2 (e.g., transfection with adhesion molecules, such as selectins or integrins, or myocardial transcription factors, such as GATA-4 or Nkx-2.5)
e.g., chronic generative myocardial disorders, such as dilatiative cardiomyopathy, or chronic ischemic cardiomyopathy: intravasal or intramyocardial administration of mRNA-transfected progenitor cells, such as CD34-positive progenitor cells or mesenchymal stem cells (e.g., transfection with adhesion molecules, such as VCAM/ICAM, or myocardial transcription factors, such as GATA-4 or Nkx-2.5)

### 2. Systemic Hematological Disorders

e.g., Leukemias: Improvement of the bone marrow homing of hematopoietic progenitor cells by means of mRNA transfection of adhesion molecules after autologous or allogenic stem cells transplantation
e.g., Leukemias: induction of cell differentiation of the malignant cells by means of mRNA transfection
mRNA transfection of differentiation factors of hematopoietic progenitor cells for differentiation in hematopoiesis with impaired cell maturation, e.g., in myelodysplastic syndrome (MDS)
mRNA transfection of inhibitory RNA for the specific blockage of translocation products of leukemia for the induction of cell differentiation

### 3. Nephrological Disorders

e.g., renal cell replacement: intravasal or intrarenal administration of mRNA-transfected progenitor cells of the kidneys, such as mesenchymal stem cells (e.g., transfection with renal transcription factors) in the treatment of chronic degenerative kidney disorders

### 4. Neurological Disorders

e.g., degenerative disorders of the nervous system, such as Parkinson's disease or Alzheimer's disease: intravasal or intracerebral administration of mRNA-transfected neuronal progenitor cells (e.g., transfection with neuronal transcription factors) in the treatment of chronic degenerative disorders of the brain

### 5. Skin Disorders

e.g., skin cell replacement after injuries/abrasions of the skin by means of intradermal or intravasal administration of mRNA-transfected dermal progenitor cells (e.g., transfection with dermal transcription factors)

### 6. Gastrointestinal Disorders

e.g., replacement of islet cells of the pancreas by means of parenchymal or intravasal administration of mRNA-transfected progenitor cells in diabetes mellitus

### 7. Orthopedic Disorders

e.g., cartilage replacement by means of parenchymal or intravasal administration of mRNA-transfected progenitor cells of the cartilage/bone.

The list below identifies a few proteins, the mRNA which codes for these proteins can be used to advantage in the method according to the present invention as mRNA that is to be transfected. Involved are adhesion molecules, i.e., molecules that allow homing of the transfected progenitor cell to the target tissue as well as cardial, hematopoietic, neuronal, renal or dermal transfection factors which promote differentiation of the transfected progenitor cells in target cells of a target tissue:

### Adhesion Molecules:

“Rolling factors:” in particular L-selectin, PSGL-1, Sialyl Lewis X on leukocytes, P- and E-selectin, GlyCAM-1, CD34, MadCAM-1 on endothelial cells and
“Adhesion molecules:” in particular integrins, such as α_{L}β₂ (LFA-1) and α_{M}β₂ (MAC-1), αₓβ₂ (p150.95), α₄β₁ (VLA-4), α₅β₁ (VLA-5) and “cellular adhesion molecules” (CAMs), such as ICAM-1, -2, VCAM-1, fibronectin on endothelial cells

### Cardiac, Hematopoietic, Neuronal, Renal, Dermal Transcription Factors:

Cardiac transcription factors: in particular Nkx-2.5, GATA-4
Hematopoietic transcription factors: in particular PU-1, CEBPα, GATA-1, CD44, CD168, p16, p15, p21, p27
Neuronal transcription factors
Renal transcription factors: in particular SalI-1, Wnt family
Dermal transcription factors: in particular Egr-1.

Below, a few examples of methods according to the present invention will be listed.

As can be seen,

FIG. 1 shows FACS analyses of mRNA versus plasmid nucleofection of hematopoietic CD34-positive human progenitor cells (HPC);

FIG. 2 shows the results of an mRNA nucleofection of CD34-positive HPC with the cardial transcription factor Nkx-2.5, and

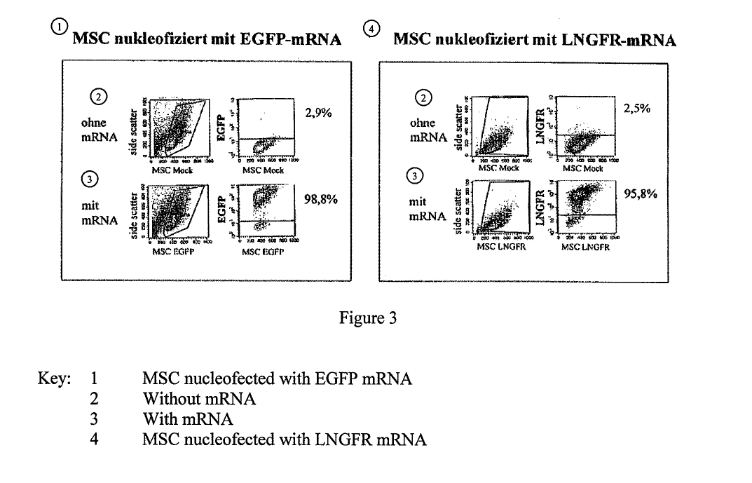

FIG. 3 shows the results of an mRNA nucleofection of mesenchymal HPC with EGFP mRNA and LNGFR mRNA.

FIG. 1 shows the results of an mRNA nucleofection and a plasmid nucleofection of hematopoietic CD34-positive human progenitor cells (HPC) with the surface markers EGFP (enhanced green fluorescent protein) and LNGFR (low-finity nerve growth factor receptor). To carry out these tests, the following methods and protocols were used:

### Cell Culture

Human CD34-positive hematopoietic progenitor cells (HPC): After G-CSF stimulation, human CD34-positive HPCs were isolated by means of leukapheresis. The immunomagnetic selection of CD34-positive cells was performed by means of the CliniMACS™ system (Miltenyi Biotech GmbH, Bergisch-Gladbach, Germany). The cells were cultured in RPMI medium (Invitrogen, Karlsruhe, Germany), supplemented with 10% FCS and the growth factors IL-3 (10 ng/mL), IL-6 (20 ng/mL), and SCF (100 ng/mL), at 37° C., 5% CO₂. The medium was changed every other day. The viability of the cells was determined by means of trypan blue staining and flow cytometry (scatter exclusion) in the standard assay.

### Human Mesenchymal Stem Cells (MSC):

Spongiosa from the human femur or tibia was harvested from volunteers between 40 and 66 years of age after having obtained their informed consent. MSCs were isolated from the bone trabecula after adhesion to positively charged plastic surfaces (NUNC, Wiesbaden, Germany) for 24 h in “complete αMEM (Cambrex, Verviers, Belgium),” supplemented with 20% heat-inactivated FBS (Gibco, Karlsruhe, Germany). Early passages (passage 2 to passage 4) were used for the experiments. After 10 to 14 days, the cells were removed from the cell culture plates by means of trypsin (Gibco) and again plated out in a cell density of 100 to 500 cells/cm². The medium was changed 2 times/week. The viability was determined by means of trypan blue absorption and flow cytometry (scatter exclusion).

### Characterization of MSC:

(a) Differentiation assay: For the differentiation assays, an initial cell count of 25,000 to 100,000 cells were plated out in cell culture flasks (NUNC), and the differentiation was induced with media of Cambrex (osteogenic and adipogenic differentiation) or Miltenyi, Bergisch-Gladbach, Germany (chondrogenic and osteogenic differentiation). To detect the differentiated cells, the cultures were fixed in 7% paraformaldehyde. (i) Osteoblasts were tested for alkaline phosphatase activity, (ii) adipogenic differentiation was tested by means of staining with saturated “Oil RedO” solution, and (iii) chondrogenic differentiation was tested by means of “alcian blue staining.” All materials for staining were purchased exclusively from SIGMA (Taufkirchen, Germany); only the “alcian blue staining kit” was obtained from Dako, Hamburg, Germany.

(b) Marker panel: Antibodies for the characterization of MSC: IgG (MOPc-21), CD3 (HIT3a), CD14 (M5E2), CD16 (3G8), CD29 (HUTS-21), CD34 (581). CD44 (G44-26), CD45 (HI30), CD73 (AD2), CD90 (5E10), CD146 (P1H12), CD166 (3A6) and CD253 (GA-R2). All antibodies were obtained from BD Pharmingen (Heidelberg, Germany), except for CD48 (J4.57, Beckman Coulter, Krefeld, Germany), CD66b (60H3, Beckman Coulter), CD105 (Sn6, Biozol-Serotec, Eching, Germany), and CD133 (293C3, Miltenyi).

### Plasmid Construction

The ΔLNGFR vector was generated by cloning the human truncated LNGFR gene into the eukaryotic pVAX1 expression vector (Invitrogen GmbH, Karlsruhe, Germany). The ΔLNGFR 834 by fragment was amplified by means of polymerase chain reaction.

### In-Vitro Transcription

The pGEM4Z/EGFP/A64 plasmid was linearized with Spe I, the pVAX/deltaLNGFR plasmid (Greiner et al. 2004, Hemother. Transf. Med.) with Xho I (New England Biolabs, Frankfurt, Germany). The linearized plasmids were purified using the “nucleotide removal kit” (Qiagen, Hilden, Germany) and used as DNA templates for the in-vitro transcription reaction. The transcription was started in a final 20 μL reaction mix at 37° C. by means of the T7 Opti-mRNA Transcription Kit (Cure Vac GmbH, Tübingen, Germany) in order to generate “5′-capped” in vitro-transcribed mRNA. The purification of the mRNA was carried out by means of DNase I digestion. To attach a poly A tail to the mRNA of delta LNGFR, a Poly(A) Tailing Kit (Ambion) was used. The mRNAs of EGFP and delta LNGFR were subsequently precipitated by means of “LiCl precipitation.” The mRNA concentration was determined by means of spectrophotometric analysis at OD₂₆₀. The RNA was stored in aliquots at −80°.

### Nucleofection

CD34-positive HPCs and MSCs were pelletized and resuspended in human CD34 Cell Nucleofector™ solutions (Amaxa GmbH, Cologne, Germany) in a cell density of 2−3×10⁶ or 5×10⁵ cells per 100 μl, The cells were nucleofected with 5 μg of mRNA or 2 μg of plasmid DNA, the programs U-08 (for HPC) or C-17 (for MSC) of the nucleofector were used. After nucleofection, the cells were immediately mixed with 500 μL of preheated culture medium and transferred into well plates with preheated medium. The cells were cultivated at 37° C. for 10 days.

### Evaluation of the Gene Expression by Means of Flow Cytometry

The delta LNGFR and EGFP expression of nucleofected and nontransfected CD34-positive HPC and MSC was determined by means of flow cytometry 1, 3, 6, 8 and 10 days after transfection. To detect delta LNGFR, the cells were incubated with “non-conjugated purified mouse monoclonal anti-human NGF antibody (Santa Cruz)” and a PE-labeled “anti-mouse IgG₁ secondary antibody (Becton Dickinson).” The data were analyzed by means of Cellquest Version 3.1 software (Becton Dickinson).

In the left column of FIG. 1A, the nucleofection with mRNA of EGFP (upper figure) and LNGFR (lower figure) is shown. It can be seen that the detection of EGFP and LNGFR in the mRNA-transfected cells gradually ends within a few days. At the beginning, however, the efficiency of the mRNA transfection is very high at 90% (EGFP/LNGFR-positive cells/total number of cells). In the plasmid nucleofection, only a nucleofection efficiency of 60 to 70% was reached (FIG. 1A, right column); however, the detection of the protein ends more slowly than in mRNA nucleofection.

FIG. 1B shows the viabilities of the nucleofected cells again for mRNA nucleofection in the left column and for plasmid nucleofection in the right column. It can be seen that the mrNA transfection leads to very high viabilities with at least 50% viable cells (both for EGFP and for LNGFR), while the viability of the transfected cell in plasmid nucleofection is very low especially at the beginning.

This indicates that the present method makes it possible to introduce suitable factors into the cells, without the disadvantages of plasmid nucleofection (genetic alteration, low viability of the altered cells, risk of tumor generation).

In FIG. 1B, the values were compared to those of a so-called “mock nucleofection,” i.e., a control in which no mRNA or plasmids were introduced into the cell.

FIG. 2 shows the results of an mRNA nucleofection of CD34-positive HPC with the cardial transcription factor Nkx-2.5. In these tests, the following additional protocol for the mRNA transfection of Nkz 2.5 by means of nucleofection was used:

5×10⁶ CD34-positive hematopoietic progenitor cells were pelletized, resuspended in 100 μL of “Human CD34 Cell Nucleofector™ Solution” (Amax GmbH, Cologne, Germany, and mixed with 5 μg of in vitro-transcribed (CureVac, Tübingen, Germany) mRNA which codes for the Nkx-2.5 protein. The cell suspension was nucleofected with the program U-08, subsequently diluted with 500 μL of preheated culture medium and transferred to 6-well plates with preheated culture medium. The cells were incubated for 4 h at 37° C., 5% CO₂, before whole protein lysates were extracted.

In FIG. 2, the expression of Nkx-2.5 appears as a band which is located between the two markers with 37.1 kD and 48.8 kD. Using this Western blot analysis, it was possible to detect the expression of Nkx-2.5 from the transfected mRNA both 4 h and 24 h after nucleofection.

FIG. 3 shows a FACS analysis of the mRNA nucleofection with EGFP mrNA and LNGFR mRNA of mesenchymal HPC. It can be seen that the efficiency of the mRNA nucleofection is between 95.8% (for LNGFR) and 98.8% (for EGFP).

1. A isolated or purified progenitor cell transfected with mRNA which codes for a protein that promotes homing of the progenitor cells in a target tissue and/or the differentiation of the progenitor cells in target cells or in cells of target tissues.

2. A pharmaceutical product comprising the progenitor cell of claim 1.

3. A method for treating a disease in a mammal comprising administering to the mammal a pharmaceutical product comprising a progenitor cell, wherein the progenitor cell has been transfected with mRNA which codes for a protein that promotes homing of the progenitor cell to a target tissue in the mammal and/or the differentiation of the progenitor cell in a target tissue, whereby the disease is treated in the mammal.

4. The method of claim 3, wherein the disease is selected from the group consisting of cardiovascular disorders, systemic hematological disorders, nephrological disorders, neurological disorders, skin disorders, gastrointestinal disorders, and orthopedic disorders.

5. A method for the targeted regeneration of a target tissue in vitro, which method comprises transfecting one or more progenitor cells with mRNA which codes for a protein that promotes homing of the progenitor cells in the target tissue and/or the differentiation of the progenitor cells in the target tissue, and introducing the transfected progenitor cells to the target tissue, whereby a target tissue is regenerated.

6. A method for the targeted regeneration of a target tissue in a patient, which method comprises transfecting one or more progenitor cells with mRNA which codes for a protein that promotes homing of the progenitor cells to the target tissue and/or the differentiation of the progenitor cells in the target tissue, and administering the transfected progenitor cells to a patient, whereby a target tissue in the patient is regenerated.

7.-8. (canceled)

9. The method of claim 6, wherein the patient is a human.

10. The method of claim 6, wherein the transfected progenitor cells are administered intravenously, intramuscularly, intracutaneously, subcutaneously, or parenchymally.

11. The method of claim 6, wherein the mRNA codes for a protein selected from the group consisting of L-selectin, PSGL-1, Sialyl Lewis X on leukocytes, P-selectin, E-selectin, GlyCAM-1, CD34, MadCAM-1, α_{L}β₂ (LFA-1) α_{M}β₂ (MAC-1), αₓβ₂, α₄β₁ (p150.95), VLA-4, α₅β₁ (VLA-5), ICAM-1, ICAM-2, VCAM-1 fibronectin, Nkx-2.5, GATA-4, PU-1, CEBPα, GATA-1, CD44, CD168, p16, p15, p21, p27, neuronal transcription factors, SalI-1, Wnt proteins, dermal transcription factors, and Egr-1.

12. The method of claim 6, wherein the target tissue is associated with a disease selected from the group consisting of cardiovascular disorders, systemic hematological disorders, nephrological disorders, neurological disorders, skin disorders, gastrointestinal disorders, and orthopedic disorders.

13. The method of claim 6, wherein the progenitor cells are autologous or allogenic progenitor cells.

14. The method of claim 6, wherein the transfecting is performed by means of electroporation or nucleotransfection.

15. The method of claim 6, wherein the progenitor cells are embryonal stem cells, nonembryonal stem cells, adult stem cells, tissue-specific adult stem cells that are specific to the target tissue or to another tissue, or other not fully differentiated cells.

16. The method of claim 6, wherein the progenitor cells are hematopoietic progenitor cells, neuronal progenitor cells, progenitor cells of the liver, progenitor cells of the skeletal muscle, progenitor cells of the skin, or cells from blood of the umbilical cord.

17. The method of claim 3, wherein the mammal is a human.

18. The method of claim 3, wherein the pharmaceutical product is administered intravenously, intramuscularly, intracutaneously, subcutaneously, or parenchymally.

19. The method of claim 3, wherein the mRNA codes for a protein selected from the group consisting of L-selectin, PSGL-1, Sialyl Lewis X on leukocytes, P-selectin, E-selectin, GlyCAM-1, CD34, MadCAM-1, α_{L}β₂ (LFA-1) α_{M}β₂ (MAC-1), αₓβ₂, α₄β₁ (p150.95), VLA-4, α₅β₁ (VLA-5), ICAM-1, ICAM-2, VCAM-1 fibronectin, Nkx-2.5, GATA-4, PU-1, CEBPα, GATA-1, CD44, CD168, p16, p15, p21, p27, neuronal transcription factors, SalI-1, Wnt proteins, dermal transcription factors, and Egr-1.

20. The method of claim 3, wherein the progenitor cells are autologous or allogenic progenitor cells.

21. The method of claim 3, wherein the transfecting is performed by means of electroporation or nucleotransfection.

22. The method of claim 3, wherein the progenitor cells are embryonal stem cells, nonembryonal stem cells, adult stem cells, tissue-specific adult stem cells that are specific to the target tissue or to another tissue, or other not fully differentiated cells.

23. The method of claim 3, wherein the progenitor cells are hematopoietic progenitor cells, neuronal progenitor cells, progenitor cells of the liver, progenitor cells of the skeletal muscle, progenitor cells of the skin, or cells from blood of the umbilical cord.

24. The isolated or purified progenitor cell of claim 1, wherein the mRNA codes for a protein selected from the group consisting of L-selectin, PSGL-1, Sialyl Lewis X on leukocytes, P-selectin, E-selectin, GlyCAM-1, CD34, MadCAM-1, α_{L}β₂ (LFA-1) α_{M}β₂ (MAC-1), αₓβ₂, α₄β₁ (p150.95), VLA-4, α₅β₁ (VLA-5), ICAM-1, ICAM-2, VCAM-1 fibronectin, Nkx-2.5, GATA-4, PU-1, CEBPα, GATA-1, CD44, CD168, p16, p15, p21, p27, neuronal transcription factors, SalI-1, Wnt proteins, dermal transcription factors, and Egr-1.

25. The isolated or purified progenitor cell of claim 1, wherein the progenitor cell is an autologous or allogenic progenitor cell.

26. The isolated or purified progenitor cell of claim 1, wherein the progenitor cell is selected from the group consisting of embryonal stem cells, nonembryonal stem cells, adult stem cells, tissue-specific adult stem cells that are specific to the target tissue or to another tissue, and other not fully differentiated cells.

27. The isolated or purified progenitor cell of claim 1, wherein the progenitor cell is selected from the group consisting of hematopoietic progenitor cells, neuronal progenitor cells, progenitor cells of the liver, progenitor cells of the skeletal muscle, progenitor cells of the skin, and cells from blood of the umbilical cord.
